(19) United States
(12) Patent Application Publication
(10) Pub. No.: US 2025/0010133 A1
(43) Pub. Date: Jan. 9, 2025

(54) SENSOR DATA TRANSMISSION DEVICE, REMOTE TERMINAL, SENSOR DATA TRANSMISSION METHOD AND PROGRAM

(71) Applicant: NIPPON TELEGRAPH AND TELEPHONE CORPORATION, Tokyo (JP)

(72) Inventor: Arinobu NIIJIMA, Tokyo (JP)

(73) Assignee: NIPPON TELEGRAPH AND TELEPHONE CORPORATION, Tokyo (JP)

(21) Appl. No.: 18/708,224

(22) PCT Filed: Dec. 15, 2021
(86) PCT No.: PCT/JP2021/046303

§ 371 Date: May 8, 2024

(51) Int. Cl. A63B 24/00; H04W 4/38

(52) U.S. Cl. CPC A63B 24/0006; H04W 4/38; A63B 2220/803; A63B 2225/20; A63B 2225/50

(57) ABSTRACT

One aspect of the present invention acquires sensor data reflecting a motion state of a measurement object at a preset sample rate, estimates a feature amount representing the motion state from the acquired sensor data, compares the estimated feature amount with a determination index prepared in advance to generate determination information of the motion state, and variably sets a transmission parameter for transmitting the sensor data to a remote place for each sample on the basis of the generated determination information.

### TECHNICAL FIELD

One aspect of the present invention relates to a sensor data transmission device, a remote terminal, a sensor data transmission method, and a program used in a system for transmitting sensor data, for example, obtained by measuring human motion from a remote side to a remote place.

### BACKGROUND ART

In recent years, various services have been proposed in which an instructor instructs movement of a human body from a remote place via a network. For example, NPL 1 describes a technique for transmitting measurement data of an acceleration sensor worn on an arm of a student to an instructor's terminal via a network, feeding back advice information based on a difference between the transmitted measurement data and acceleration data corresponding to a model movement from the instructor's terminal to the student's terminal, and notifying the student of the advice information through auditory or tactile sensation. By using this type of service, the student can receive support of exercise instruction or the like from the instructor while staying at home or the like.

### CITATION LIST

### Non Patent Literature

[NPL 1] Villa, Steeven, et al. “Assisting Motor Skill Transfer for Dance Students Using Wearable Feedback.” 2021
International Symposium on Wearable Computers. 2021.

### SUMMARY OF INVENTION

### Technical Problem

When the above-described remote exercise support service is implemented, it is necessary to transmit data obtained by measuring the movement of the body of the student at a high transmission rate. However, if the measurement data is transmitted at a high transmission rate, the power consumption of the remote-side device increases, and as a result, the battery life becomes short, and it becomes difficult to receive sufficient support. On the other hand, if the transmission rate of the measurement data is suppressed to be low in order to reduce the power consumption, the real-time performance of the exercise support is impaired, resulting in a decrease in the effects of exercise support.

The present invention has been made in view of the above circumstances, and an object of the present invention is to provide a technique for reducing power consumption of a remote-side device while maintaining real-time performance of support processing performed by the remote place side.

### Solution to Problem

In order to solve the above problem, according to one aspect of the present invention, there is provided a sensor data transmission device or a sensor data transmission method including: a first processing unit configured to acquire or a step of acquiring sensor data reflecting a motion state of a measurement object at a preset sample rate; a second processing unit configured to estimate or a step of estimating a feature amount representing the motion state from the sensor data; a third processing unit configured to compare or a step of comparing the feature amount with a determination index prepared in advance to generate determination information of the motion state; and a fourth processing unit configured to variably set or a step of variably setting a transmission parameter for transmitting the sensor data to a remote place for each sample on the basis of the determination information.

According to one aspect of the present invention, for example, in order to receive exercise support, when transmitting sensor data reflecting a motion state of a user to a remote place, it is determined whether the motion state of the user is good or poor on the basis of a feature amount of the motion state estimated from the sensor data, and a transmission parameter for the sensor data, such as a transmission rate or an amount of transmission data, is variably set on the basis of the determination result. For example, when the motion state of the user is deteriorating, the sensor data is transmitted at a high transmission rate or with a sufficient amount of data, while when the motion state of the user is good, the sensor data is transmitted at a low transmission rate or with a limited small amount of data.

Therefore, when the motion state of the user is good, the sensor data is transmitted at a low speed or in a state where the amount of data is limited, and thus the power consumption of the sensor data transmission device is reduced and the battery life can be extended. On the other hand, when the motion state of the user is deteriorating, the sensor data is transmitted to the remote place at a high speed and with a sufficient amount of data, and thus the motion state of the user can be evaluated in real time and with high accuracy on the basis of the sensor data at the remote place.

### Advantageous Effects of Invention

That is, according to one aspect of the present invention, it is possible to provide a technique for reducing power consumption of a remote-side device while maintaining real-time performance of support processing performed by the remote place side.

### BRIEF DESCRIPTION OF DRAWINGS

FIG. 1 is a diagram illustrating an example of a configuration of a remote exercise support system according to one embodiment of the present invention.

FIG. 2 is a block diagram illustrating an example of a hardware configuration of a sensor data transmission device according to one embodiment of the present invention.

FIG. 3 is a block diagram illustrating an example of a software configuration of the sensor data transmission device according to one embodiment of the present invention.

FIG. 4 is a flowchart illustrating an example of a processing procedure and processing content of sensor data transmission control executed by a control unit of the sensor data transmission device illustrated in FIG. 3.

FIG. 5 is a diagram illustrating an example of an acceleration norm when a user's gait is deteriorating in comparison with an acceleration norm of a target gait.

FIG. 6 is a diagram illustrating an example of an acceleration norm when a user's gait is good in comparison with an acceleration norm of a target gait.

FIG. 7 is a diagram illustrating a change in a remaining battery amount when communication rate control is performed in comparison with a change in a remaining battery amount when the communication rate is made constant.

FIG. 8 is a diagram illustrating an example of communication rate control results in another embodiment of the present invention.

### DESCRIPTION OF EMBODIMENTS

Embodiments of the present invention will be described below with reference to the drawings.

### One Embodiment

### (Configuration Example)

### (1) System

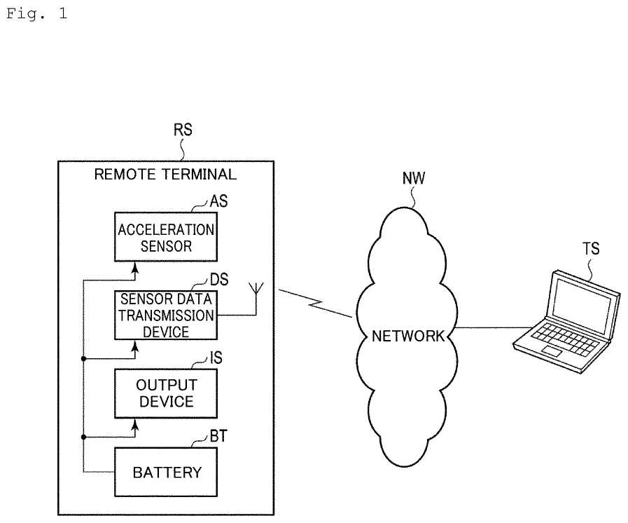

FIG. 1 is a diagram illustrating an example of a configuration of a remote exercise support system according to one embodiment of the present invention. The remote exercise support system enables data transmission via a network NW between a remote terminal RS used by a user who receives exercise support and an instructor terminal TS for performing exercise support.

The remote terminal RS includes an acceleration sensor AS, a sensor data transmission device DS, an output device IS, and a battery BT. The acceleration sensor AS is worn, for example, on a predetermined part of a user's body, for example, on the waist, measures a triaxial acceleration waveform of a motion related to the user's exercise, and outputs acceleration data representing the measured acceleration waveform to the sensor data transmission device DS.

The output device IS is for notifying the user of advice information of the instructor for the exercise of the user sent from the instructor terminal TS by audio or vibration, and is constituted by, for example, an earphone or a vibrator.

The battery BT is composed of, for example, a lithium ion polymer battery, and supplies an operating voltage to the acceleration sensor AS, the sensor data transmission device DS, and the output device IS directly or via the sensor data transmission device DS.

In one embodiment, a case where the remote terminal RS is a wearable terminal incorporating the acceleration sensor AS, the sensor data transmission device DS, and the output device IS will be described as an example. However, in addition, for example, the function of the sensor data transmission device DS may be provided in a smartphone, the acceleration sensor AS and the output device IS may be constituted by independent devices, and they may be connected to the smartphone via a wireless interface such as a signal cable or Bluetooth (registered trademark).

The instructor terminal TS is composed of an information processing device such as a personal computer or a server computer. The instructor terminal TS has a function of comparing acceleration data of a motion related to the exercise of the user sent from the remote terminal RS with acceleration data corresponding to a motion to be a model of the exercise of the instructor to obtain a difference, generating advice information corresponding to the difference, and transmitting the advice information to the remote terminal.

The network NW includes, for example, a wide area network centered on the Internet and an access network for accessing the wide area network. As the access network, for example, a public communication network using wired or wireless communication or a wireless local area network (LAN) is used.

### (2) Sensor Data Transmission Device DS

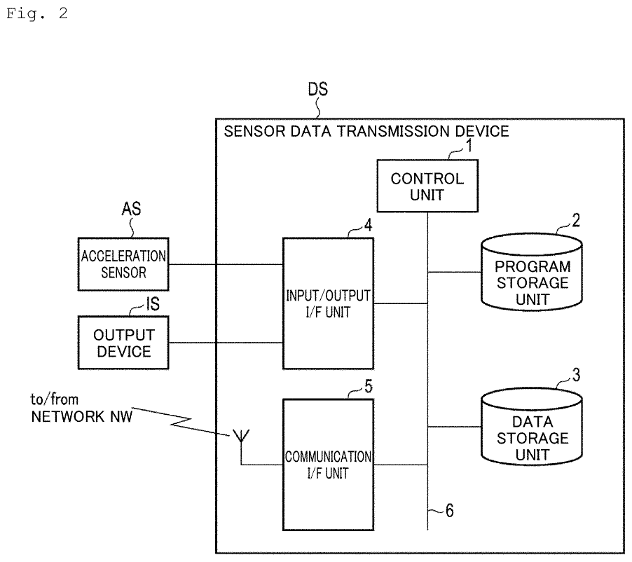

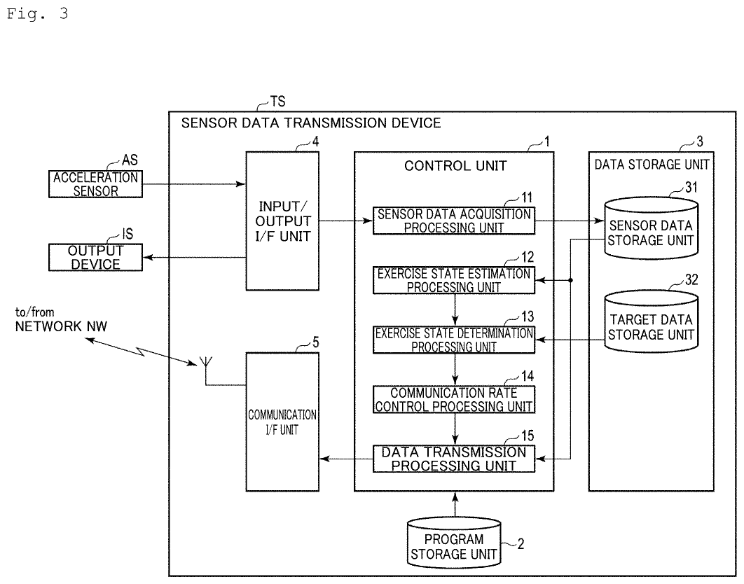

FIGS. 2 and 3 are block diagrams respectively illustrating an example of hardware and software configurations of the sensor data transmission device DS according to one embodiment of the present invention.

The sensor data transmission device DS includes a control unit 1 using a hardware processor such as a central processing unit (CPU). A storage unit having a program storage unit 2 and a data storage unit 3, an input/output interface (hereinafter, interface is referred to as I/F) unit 4, and a communication I/F unit 5 are connected to the control unit 1 via a bus 6.

The communication I/F unit 5 performs data communication with the instructor terminal TS according to a communication protocol defined by the network NW under the control of the control unit 1.

The acceleration sensor AS and the output device IS are connected to the input/output I/F unit 4. The input/output I/F unit 4 has a function of receiving acceleration data output from the acceleration sensor AS and a function of receiving advice information sent from the instructor terminal TS from the control unit 1 and outputting the advice information to an output device IS.

The program storage unit 2 is configured, for example, by combining a non-volatile memory such as a solid state drive (SSD) that can be written and read at any time and a non-volatile memory such as a read only memory (ROM). In addition to middleware such as an operating system (OS), the program storage unit 2 stores application programs necessary for executing various kinds of control processing according to one embodiment. Hereinafter, the OS and each application program will be collectively referred to as programs.

The data storage unit 3 is constituted by, for example, a non-volatile memory such as an SSD that can be written and read at any time, and a volatile memory such as a random access memory (RAM). The data storage unit 3 is provided with a sensor data storage unit 31 and a target data storage unit 32 as main storage units necessary for carrying out one embodiment.

The sensor data storage unit 31 temporarily stores the acceleration data acquired from the acceleration sensor AS for data transmission control processing to be described later.

The target data storage unit 32 is used for storing target data used for determining the quality of the user's exercise estimated from the acceleration data. The target data may be stored in advance in the target data storage unit 32, but may be stored by receiving target data corresponding to the movement to be a model of the instructor from the instructor terminal TS during the user's exercise, for example. As the target data, it is desirable to prepare a plurality of pieces of target data created for each user attribute such as the age group, sex, height, and weight of the user.

The control unit 1 includes a sensor data acquisition processing unit 11, an exercise state estimation processing unit 12, an exercise state determination processing unit 13, a communication rate control processing unit 14, and a data transmission processing unit 15 as processing functions necessary for carrying out one embodiment. These processing units 11 to 15 are implemented by causing the hardware processor of the control unit 1 to execute an application program stored in the program storage unit 2.

The control unit 1 may be configured using a programmable logic device (PLD), a field programmable gate array (FPGA), or the like. Further, the application program may be downloaded from an application server (not illustrated) or the like and stored in the program storage unit 2 when necessary, in addition to being stored in the program storage unit 2 in advance.

The sensor data acquisition processing unit 11 acquires acceleration data output from the acceleration sensor AS at a predetermined sample rate via the input/output I/F unit 4 during an exercise state determination period, and performs processing for storing the acquired acceleration data in the sensor data storage unit 31 in association with information indicating a measurement time or an acquisition time for each sample.

The exercise state estimation processing unit 12 reads the acceleration data from the sensor data storage unit 31 by a predetermined number of samples, and performs, for example, fast Fourier transform on the read acceleration data. Then, processing for extracting and outputting a peak value of a frequency component as a feature amount of an exercise state of a user from data obtained by the fast Fourier transform is performed.

The exercise state determination processing unit 13 receives the peak value from the exercise state estimation processing unit 12, and compares the received peak value with target data stored in the target data storage unit 32. Then, it is determined whether or not the peak value is equal to or less than the target data, and processing for outputting the determination result as information indicating a determination result as to whether the exercise state is good or poor is performed.

The communication rate control processing unit 14 receives information indicating the determination result of the exercise state from the exercise state determination processing unit 13, and performs processing for setting a communication rate of acceleration data of a sample corresponding to time on the basis of the received information indicating the determination result of the exercise state.

The data transmission processing unit 15 reads the acceleration data from the sensor data storage unit 31 for each sample, and performs processing for transmitting the read acceleration data from the communication I/F 5 to the instructor terminal TS according to the communication rate set by the communication rate control processing unit 14.

### (Operation Example)

Next, an operation example of the sensor data transmission device DS configured as described above will be described. In this example, a case will be described where the acceleration data related to the walking exercise of the user is transmitted from the sensor data transmission device DS to the instructor terminal TS. Further, the description will be made on the assumption that the target data of the walking exercise is stored in the target data storage unit 32 in advance.

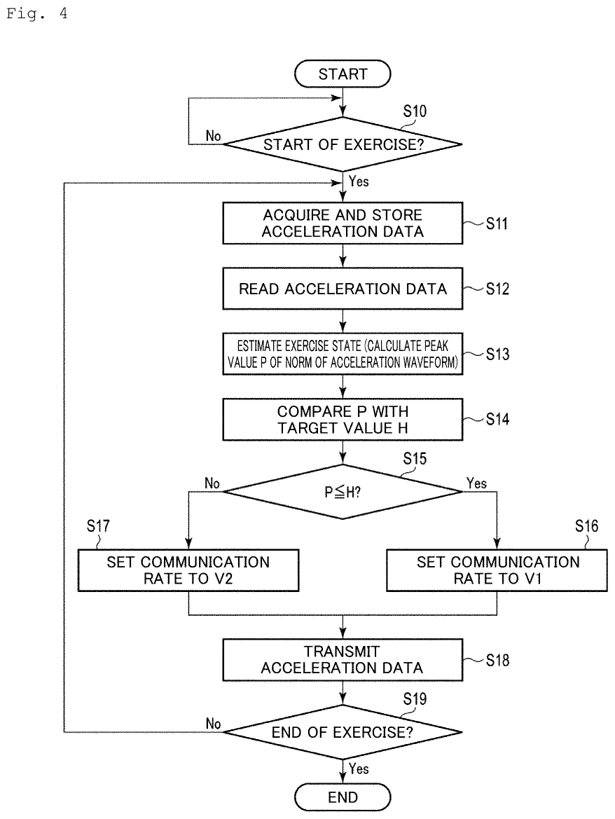

FIG. 4 is a flowchart illustrating an example of a processing procedure and processing content of sensor data transmission control executed by the control unit 1 of the sensor data transmission device DS.

### (1) Acquisition of Sensor Data

The control unit 1 of the sensor data transmission device DS monitors the start of the walking exercise of the user in step S10. In this state, it is assumed that the user wears the remote terminal RS on the waist, for example, and then starts walking exercise by activating the exercise support application of the remote terminal RS. Then, under the control of the sensor data acquisition processing unit 11, the control unit 1 of the sensor data transmission device DS acquires acceleration data output from the acceleration sensor AS at a predetermined sample rate via the input/output I/F unit 4 in step S11. The acquired acceleration data is stored in the sensor data storage unit 31 in association with the measurement time or the acquisition time for each sample. Here, the acceleration data includes triaxial acceleration waveform data. The gait of the user, that is, the stride, the walking period, or the like, is expressed by the triaxial acceleration waveform data.

### (2) Estimation of Exercise State

Each time a predetermined sample of the acceleration data is stored in the sensor data storage unit 31, the control unit 1 of the sensor data transmission device DS reads the acceleration data of the predetermined sample (for example, 64 samples) from the sensor data storage unit 31 in step S12 under the control of the exercise state estimation processing unit 12.

Subsequently, the exercise state estimation processing unit 12 performs fast Fourier transform processing on the acceleration data of the predetermined sample in step S13, and thereby obtains data on a frequency axis. The exercise state estimation processing unit 12 detects a peak value of the data on the frequency axis, and outputs the detected peak value as information indicating a feature amount of walking exercise of the user.

For example, the exercise state estimation processing unit 12 calculates a norm (√(x²+y²+z²)) of amplitude values (x, y, z) of triaxial acceleration waveform data included in acceleration data, and uses a peak value P of the calculated norm as a feature amount representing a walking exercise state of the user. The peak value P represents the magnitude of an impact when the user's heel contacts the ground during walking.

### (3) Determination of Exercise State

Subsequently, under the control of the exercise state determination processing unit 13, the control unit 1 of the sensor data transmission device DS compares the peak value P obtained by the exercise state estimation processing unit 12 in step S14 with a target value H corresponding in time stored in the target data storage unit 32. Then, in step S15, the exercise state determination processing unit 13 determines whether or not the peak value P is equal to or less than the target value H on the basis of the comparison result, and outputs the determination result to the communication rate control processing unit 14.

Here, the state in which the estimated peak value P is equal to or less than the target value H (P≤H) corresponds to a state in which the magnitude of the impact when the user's heel contacts the ground during walking does not reach the target value, that is, a “poor state” in which the user does not walk with his/her legs fully raised.

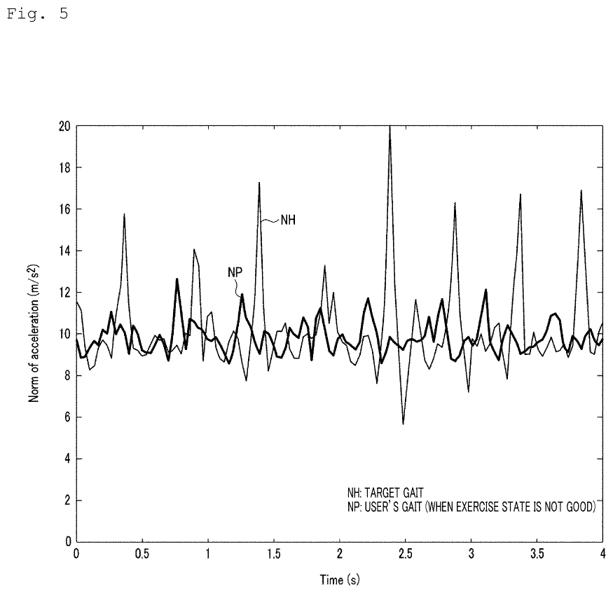

FIG. 5 illustrates an example of a case in which the temporal change of the norm of the acceleration waveform obtained when the walking exercise state of the user is not good is displayed in such a manner that the time axis is aligned with the change of the norm of the acceleration waveform due to the target walking exercise. As illustrated in FIG. 5, the norm NP of the acceleration waveform when the user's walking exercise is not good has a lower waveform peak value than the norm NH of the acceleration waveform corresponding to the target walking exercise.

On the other hand, when the peak value P is larger than the target value H (P>H), it corresponds to a state in which the magnitude of the impact when the user's heel contacts the ground during walking is larger than the target value, that is, a “good state” in which the user walks with his/her legs fully raised.

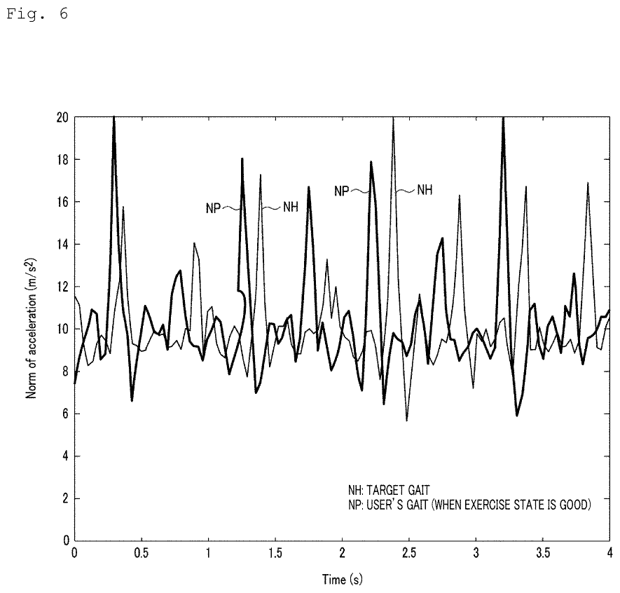

FIG. 6 illustrates an example of a case in which the temporal change of the norm of the acceleration waveform obtained when the walking exercise state of the user is good is displayed in such a manner that the time axis is aligned with the change of the norm of the acceleration waveform due to the target walking exercise. As illustrated in FIG. 6, the norm NP of the acceleration waveform when the walking exercise state of the user is good has a higher waveform peak value than the norm NH of the acceleration waveform corresponding to the target walking exercise.

The exercise state determination processing unit 13 determines whether the walking exercise state is good or poor on the basis of the difference of the peak values of the norm of the acceleration waveform.

### (4) Variable Setting of Communication Rate

Next, under the control of the communication rate control processing unit 14, the control unit 1 of the sensor data transmission device DS variably sets the communication rate when transmitting the acceleration data in the following manner on the basis of the determination result of the walking motion.

That is, if the peak value P is equal to or less than the target value H (P≤H) as a result of the determination, the communication rate control processing unit 14 determines that the walking exercise state of the user is in a “poor state,” and proceeds to step S16. In this case, when the walking motion state of the user is in a “poor state,” it is necessary to transmit the acceleration data to the instructor terminal TS at a high speed without omitting the acceleration data so that the instructor can perform the walking instruction with high accuracy and in real time. Therefore, the communication rate control processing unit 14 sets the communication rate of the acceleration data to a first communication rate V1 set high in step S16.

On the other hand, when the peak value P is larger than the target value H (P>H), the communication rate control processing unit 14 determines that the walking exercise state of the user is in a “good state” and proceeds to step S17. Here, when the walking exercise state of the user is in a “good state,” it is not necessary for the instructor to perform walking instruction to the user in real time. That is, it is not necessary to transmit the acceleration data to the instructor terminal TS at a high speed. Therefore, the communication rate control processing unit 14 sets the communication rate of the acceleration data to a second communication rate V2 set lower than the first communication rate V1 in step S17. It is also possible to set the second communication rate V2 to zero, that is, not to transmit the acceleration data, when the walking exercise state of the user is in a “good state.”

### (5) Transmission of Acceleration Data

Under the control of the data transmission processing unit 15, the control unit 1 of the sensor data transmission device DS performs the transmission processing of the acceleration data in step S18 as follows. That is, the data transmission processing unit 15 receives the set communication rate from the communication rate control processing unit 14, and reads the acceleration data of the sample corresponding to the time from the sensor data storage unit 31. Then, the data transmission processing unit 15 transmits the read acceleration data from the communication I/F unit 5 to the instructor terminal TS via the network NW according to the communication rate.

In the instructor terminal TS, for example, the acceleration data transmitted from the remote terminal RS is compared with acceleration data corresponding to a model walking exercise, and advice information for the walking exercise of the user is generated on the basis of the difference. As the advice information, in the case of walking exercise, for example, information for improving the gait of the user such as widening a stride, shortening a walking cycle, and raising his/her legs slightly higher is used. The generated advice information is transmitted from the instructor terminal TS to the remote terminal RS via the network NW, output as audio information or vibration information from the output device IS of the remote terminal RS, and is notified to the user.

The control unit 1 of the sensor data transmission device DS repeatedly executes a series of data transmission processing in steps S11 to S19 until the end of the walking exercise of the user is detected in step S19.

### (Action and Effects)

As described above, in the sensor data transmission device DS according to one embodiment, control processing for data transmission is performed as follows. That is, acceleration data measured during walking exercise of the user is acquired for each sample, a peak value of a norm of a triaxial acceleration waveform is detected as a feature amount of the walking exercise from the acquired acceleration data, and the peak value is compared with preset target data to determine whether the walking exercise state is good or poor. When it is determined that the walking exercise state is not good, the communication rate is set to a first communication rate V1 which is high, and acceleration data corresponding to the time is transmitted at a high speed. On the other hand, when it is determined that the walking exercise state is good, the communication rate is set to a second communication rate V2 lower than the first communication rate V1, and acceleration data corresponding to the time is transmitted at a low speed.

Therefore, according to one embodiment, when the walking exercise state of the user is not good, the acceleration data at that time is transmitted at a high speed without omission. Therefore, the instructor terminal TS can evaluate the walking exercise state of the user accurately and in real time on the basis of the acceleration data, and can notify the user of effective advice information at appropriate timing.

On the other hand, when the walking exercise state of the user is good, the acceleration data at that time is transmitted at a low speed or thinned out and transmitted because accurate instruction is not required in real time. As a result, the power consumption of the sensor data transmission device DS is suppressed to be small, and the life of the battery BT of the remote terminal RS can be extended, thereby making it possible to fully use the remote terminal RS even when exercising for a long time.

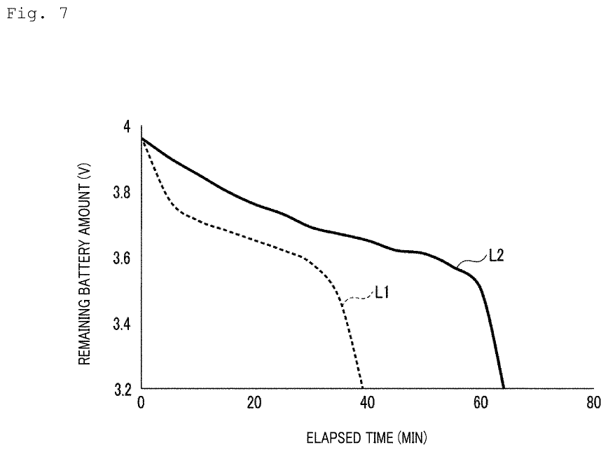

FIG. 7 illustrates an example of measurement results of changes in the remaining amount of the battery BT of the remote terminal RS from 4 V (fully charged) to 3.2 V (battery exhausted) in a case L1 where the communication rate is fixed to 100 Hz and a case L2 where the communication rate is variably set on the basis of a determination result of a walking exercise state. As illustrated in FIG. 7, it was confirmed that by variably setting the communication rate, the life of the battery BT can be extended by about 1.5 times compared with the case where the communication rate is fixed.

### Other Embodiments

(1) In one embodiment, the case where the magnitude relationship between the peak value P of the norm of the acceleration waveform and the target value H is determined in the exercise state determination processing, and the communication rate is set to either the first communication rate V1 or the second communication rate V2 lower than the first communication rate V1 on the basis of the determination result has been described as an example.

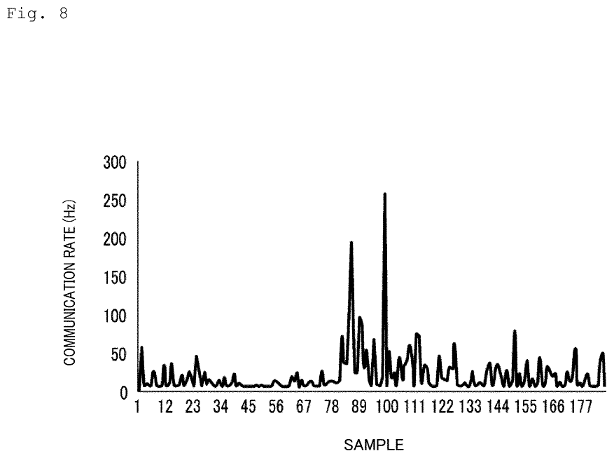

However, the present invention is not limited to the above embodiment, and for example, when the peak value P of the norm of the acceleration waveform is equal to or less than the target value H, the difference may be calculated, and the communication rate may be variably set in a stepless manner or in a plurality of stages for each sample according to the calculated difference value. FIG. 8 illustrates an example of setting the communication rate in the case where the communication rate is variably set in a stepless manner or in a plurality of stages.

Also, when the peak value P of the norm of the acceleration waveform is larger than the target value H, the difference may be calculated, the communication rate may be set to V2 when the calculated difference value is equal to or less than a preset threshold value, and the communication rate may be set to zero, that is, not transmitted, when the difference value is equal to or greater than the threshold value.

(2) In one embodiment, the case where the communication rate of the acceleration data, that is, the transmission rate of the acceleration data, is variably set according to the determination result of the walking exercise state has been described. However, the present invention is not limited thereto, and the data compression rate for the acceleration data may be variably set according to, for example, the determination result of the walking exercise state, thereby varying the transmission data amount of the transmission data.

For example, when the walking exercise state is not good, the acceleration data are transmitted as they are without thinning out, while when the walking exercise state is good, the acceleration data are appropriately thinned out to reduce the amount of transmission data. Thus, the transmission time of the acceleration data when the walking exercise state is good is shortened, and the power consumption in the sensor data transmission device DS can be reduced accordingly.

(3) In one embodiment, the case where the user performs walking exercise has been described as an example, but the motion of the body in accordance with various physical exercises and training other than walking exercise may be measured by the acceleration sensor, the exercise state of the user may be determined on the basis of the measured acceleration data, and the communication rate may be variably set on the basis of the determination result.

As a sensor for detecting the motion of the user, a myoelectric sensor or the like for measuring the movement of the muscle may be used in addition to the acceleration sensor. Further, the movement of the user's body may be indirectly measured using a vital sensor for detecting an electrocardiogram, a respiration rate, a blood pressure, and the like.

(4) In one embodiment, the case of determining the exercise state of a human has been described as an example, but the motion state of a robot or an animal may be determined in addition, and the functional configuration, processing procedures, processing contents, and the like of the sensor data transmission device may be variously modified and implemented without departing from the gist of the present invention.

Although the embodiments of the present invention have been described in detail above, the above description is merely illustrative of the present invention in every respect. It goes without saying that various modifications and variations can be made without departing from the scope of the present invention. That is, a specific configuration according to the embodiment may be appropriately employed in implementing the present invention.

This invention is not limited to the embodiment as it is but can be embodied by modifying components in the practical phase without departing from the gist thereof. Further, various inventions can be formed by appropriate combinations of the plurality of components disclosed in the above embodiments. For example, some components of all the components shown in the embodiment may be omitted. Furthermore, components of different embodiments may be combined as appropriate.

### REFERENCE SIGNS LIST

RS Remote terminal
AS Acceleration sensor
DS Sensor data transmission device
IS Output device
BT Battery
NW Network
TS Instructor terminal
1 Control unit
2 Program storage unit
3 Data storage unit
4 Input/output I/F unit
5 Communication I/F unit
6 Bus
11 Sensor data acquisition processing unit
12 Exercise state estimation processing unit
13 Exercise state determination processing unit
14 Communication rate control processing unit
15 Data transmission processing unit
31 Sensor data storage unit
32 Target data storage unit

1. A sensor data transmission device, comprising:
first processing circuitry configured to acquire sensor data reflecting a motion state of a measurement object at a preset sample rate;
second processing circuitry configured to estimate a feature amount representing the motion state from the sensor data;
third processing circuitry configured to compare the feature amount with a determination index prepared in advance to generate determination information of the motion state; and
fourth processing circuitry configured to variably set a transmission parameter for transmitting the sensor data to a remote place for each sample on the basis of the determination information.

2. The sensor data transmission device according to claim 1, wherein:
the third processing circuitry determines whether the motion state is in a first state in which the motion state is deteriorating or in a second state in which the motion state is good by comparing the feature amount with a threshold value set as the determination index, and
the fourth processing circuitry sets at least one of a transmission rate and an amount of transmission data as the transmission parameter to a first value when the motion state is determined to be the first state, and sets at least one of the transmission rate and the amount of transmission data to a second value lower than the first value when the motion state is determined to be the second state.

3. The sensor data transmission device according to claim 1, wherein:
the third processing circuitry calculates a difference between the feature amount representing the motion state and a target value set as the determination index, and
the fourth processing circuitry variably sets the transmission parameter on the basis of the difference such that at least one of a transmission rate and an amount of transmission data decreases as the difference decreases.

4. The sensor data transmission device according to claim 1, wherein:
the second processing circuitry calculates a peak value of a norm of the sensor data as the feature amount representing the motion state.

5. The sensor data transmission device according to claim 1, further comprising:
a fifth processing circuitry configured to transmit the sensor data for each sample according to the transmission parameter.

6. A remote terminal comprising:
the sensor data transmission device according to claim 1; and
a sensor to measure the motion state of the measurement object and output the sensor data reflecting the motion state to the sensor data transmission device.

7. A sensor data transmission method, comprising:
acquiring sensor data reflecting a motion state of a measurement object at a preset sample rate;
estimating a feature amount representing the motion state from the sensor data;
comparing the feature amount with a determination index prepared in advance to generate determination information of the motion state; and
variably setting a transmission parameter for transmitting the sensor data to a remote place for each sample on the basis of the determination information.

8. A non-transitory computer readable medium storing a program for causing a processor to perform the method of claim 7.
